(19) United States
(12) Patent Application Publication
(10) Pub. No.: US 2023/0293358 A1
(43) Pub. Date: Sep. 21, 2023

(54) Menstrual Pad Assembly

(71) Applicant: Gernai Peyrefitte, San Bernardino, CA (US)

(72) Inventor: Gernai Peyrefitte, San Bernardino, CA (US)

(21) Appl. No.: 17/697,277

(22) Filed: Mar. 17, 2022

(51) Int. Cl. A61F 13/472; A61F 13/47; A61F 13/535

(52) U.S. Cl. CPC A61F 13/47227; A61F 13/4702; A61F 13/535; A61F 2013/530802

(57) ABSTRACT

A menstrual pad assembly for absorbing menstrual fluids includes a menstrual pad that has a first portion which is perpendicularly oriented with a second portion such that the menstrual pad forms a plus shape. An absorbent member extends upwardly from the second portion of the menstrual pad to extend between a user’s genitalia and the user’s buttocks when the menstrual pad is worn. The absorbent member absorbs bodily fluids thereby inhibiting the bodily fluids from escaping the menstrual pad. A ridge extends upwardly from the second portion of the menstrual pad to surround the user’s genitalia when the menstrual pad is worn. The ridge is comprised of a fluid absorbent material to inhibit bodily fluids from escaping the second portion.

### (B) CROSS-REFERENCE TO RELATED APPLICATIONS

Not Applicable

### (C) STATEMENT REGARDING FEDERALLY SPONSORED RESEARCH OR DEVELOPMENT

Not Applicable

### (D) THE NAMES OF THE PARTIES TO A JOINT RESEARCH AGREEMENT

Not Applicable

### (E) INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### (F) STATEMENT REGARDING PRIOR DISCLOSURES BY THE INVENTOR OR JOINT INVENTOR

Not Applicable

### (G) BACKGROUND OF THE INVENTION

### Field of the Invention

The disclosure relates to menstrual devices and more particularly pertains to a new menstrual device for absorbing menstrual fluids. The device includes a menstrual pad that includes an absorbent member which extends upwardly from the menstrual pad such that the absorbent member extends between the user’s genital and the user’s buttocks. The device includes an absorbent ridge that extends upwardly from said menstrual pad such that the absorbent ridge surrounds the user’s genitalia. In this way the absorbent member and the absorbent ridge inhibit menstrual fluids from leaking past the menstrual pad when the user lies down.

### Description of Related Art Including Information Disclosed Under 37 CFR 1.97 and 1.98.

The prior art relates to menstrual devices including a sanitary napkin that includes a raised absorbent element to reduce leakage of bodily fluids. The prior art discloses a sanitary napkin that includes a projection that is temporarily fixed to the sanitary napkin. The prior art discloses a sanitary napkin that includes an ovoid raised portion and a rectangular raised portion for reducing leakage of bodily fluids.

### (H) BRIEF SUMMARY OF THE INVENTION

An embodiment of the disclosure meets the needs presented above by generally comprising a menstrual pad that has a first portion which is perpendicularly oriented with a second portion such that the menstrual pad forms a plus shape. An absorbent member extends upwardly from the second portion of the menstrual pad to extend between a user’s genitalia and the user’s buttocks when the menstrual pad is worn. The absorbent member absorbs bodily fluids thereby inhibiting the bodily fluids from escaping the menstrual pad. A ridge extends upwardly from the second portion of the menstrual pad to surround the user’s genitalia when the menstrual pad is worn. The ridge is comprised of a fluid absorbent material to inhibit bodily fluids from escaping the second portion.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### I BRIEF DESCRIPTION OF SEVERAL VIEWS OF THE DRAWINGS

The disclosure will be better understood and objects other than those set forth above will become apparent when consideration is given to the following detailed description thereof. Such description makes reference to the annexed drawings wherein:

FIG. 1 is a top perspective view of a menstrual pad assembly according to an embodiment of the disclosure.

FIG. 2 is a top view of an embodiment of the disclosure.

FIG. 3 is a right side view of an embodiment of the disclosure.

### (J) DETAILED DESCRIPTION OF THE INVENTION

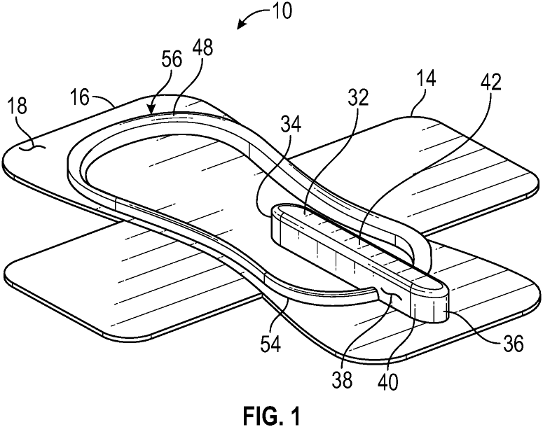

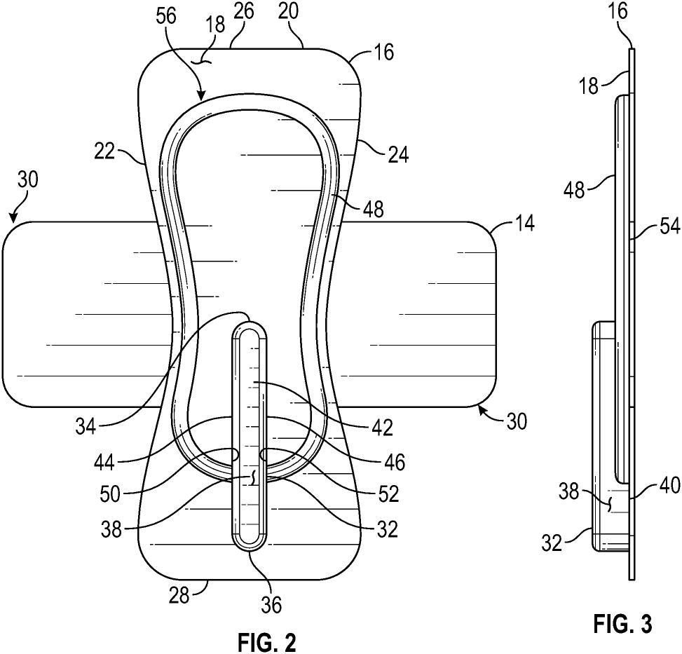

With reference now to the drawings, and in particular to FIGS. 1 through 3 thereof, a new menstrual device embodying the principles and concepts of an embodiment of the disclosure and generally designated by the reference numeral 10 will be described.

As best illustrated in FIGS. 1 through 3, the menstrual pad assembly 10 generally comprises a menstrual pad 12 that has a first portion 14 which is perpendicularly oriented with a second portion 16 such that the menstrual pad 12 forms a plus shape. The second portion 16 has a top surface 18 and a perimeter edge 20, and the perimeter edge 20 has a first lateral side 22, a second lateral side 24, a front side 26 and a back side 28. The second portion 16 is elongated between the front side 26 and the back side 28, and each of the first lateral side 22 and the second lateral side 24 curves inwardly toward a center of the second portion 16. The first portion 14 extends outwardly from each of the first lateral side 22 and the second lateral side 24 such that the first portion 14 defines a pair of wings 30 of the menstrual pad 12. Each of the first portion 14 and the second portion 16 are comprised of a fluid absorbent material such that each of the first portion 14 and the second portion 16 can absorb bodily fluids.

An absorbent member 32 extends upwardly from the second portion 16 of the menstrual pad 12. The absorbent member 32 is oriented to extend along a longitudinal axis of the second portion 16 such that the absorbent member 32 extends between a user’s genitalia and the user’s buttocks when the menstrual pad 12 is worn. The absorbent member 32 is comprised of a fluid absorbent material to absorb bodily fluids thereby inhibiting the bodily fluids from escaping the menstrual pad 12. The absorbent member 32 has a first end 34, a second end 36 and an outer surface 38 extending between the first end 34 and the second end 36. The outer surface 38 has a lower side 40, an upper side 42, a first sidelong side 44 and a second sidelong side 46.

The lower side 40 is bonded to the top surface 18 of the second portion 16 and the absorbent member 32 is elongated between the first end 34 and the second end 36. The absorbent member 32 is oriented to extend along an axis extending between the front side 26 and the back side 28 of the perimeter edge 20 of the second portion 16. Furthermore, the absorbent member 32 is centrally positioned between the first lateral side 22 and the second lateral side 24 of the perimeter edge 20 of the second portion 16. The second end 36 of the absorbent member 32 is positioned adjacent to the back side 28 of the perimeter edge 20 of the second portion 16, and each of the first end 34 and the second end 36 of the absorbent member 32 is rounded.

A ridge 48 extends upwardly from the second portion 16 of the menstrual pad 12. The ridge 48 forms a closed loop to surround the user’s genitalia when the menstrual pad 12 is worn. The ridge 48 is comprised of a fluid absorbent material to inhibit bodily fluids from escaping the second portion 16 thereby inhibiting the bodily fluids from staining clothing worn by the user. The ridge 48 has a primary end 50, a secondary end 52 and a lowermost surface 54 extending between the primary end 50 and the secondary end 52. The lowermost surface 54 is bonded to the top surface 18 of the second portion 16 and each of the primary end 50 and the secondary end 52 abuts a respective one of the first sidelong side 44 and the second sidelong side 46 of the outer surface 38 of the absorbent member 32 at a point located adjacent to the second end 36 of the absorbent member 32. Furthermore, the ridge 48 follows curvature of each of the first lateral side 22 and the second lateral side 24 of the perimeter edge 20 of the second portion 16. Additionally, the ridge 48 forms a loop 56 that is positioned adjacent to the front side 26 of the perimeter edge 20 of the second portion 16.

In use, the menstrual pad 12 is worn such that the ridge 48 surrounds the user’s genitalia and the absorbent member 32 extends between the user’s genitalia and the user’s buttocks. In this way the ridge 48 and the absorbent member 32 absorb bodily fluids that commonly leak from existing menstrual pads when the user lies down to sleep. Thus, the bodily fluids are inhibited from staining clothing or bedding while the user is sleeping. The menstrual pad 12 is discarded in the conventional manner of existing menstrual pads.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A menstrual pad assembly for inhibiting leakage when a user is lying down, said assembly comprising:
a menstrual pad having a first portion being perpendicularly oriented with a second portion such that said menstrual pad forms a plus shape;
an absorbent member extending upwardly from said second portion of said menstrual pad, said absorbent member being oriented to extend along a longitudinal axis of said second portion wherein said absorbent member is configured to extend between a user’s genitalia and the user’s buttocks when said menstrual pad is worn, said absorbent member being comprised of a fluid absorbent material wherein said absorbent member is configured to absorb bodily fluids thereby inhibiting the bodily fluids from escaping said menstrual pad; and
a ridge extending upwardly from said second portion of said menstrual pad, said ridge forming a closed loop wherein said ridge is configured to surround the user’s genitalia when said menstrual pad is worn, said ridge being comprised of a fluid absorbent material wherein said ridge is configured to inhibit bodily fluids from escaping said second portion thereby inhibiting the bodily fluids from staining clothing worn by the user.

2. The assembly according to claim 1, wherein said second portion has a top surface and a perimeter edge, said perimeter edge having a first lateral side, a second lateral side, a front side and a back side, said second portion being elongated between said front side and said back side, each of said first lateral side and said second lateral side curving inwardly toward a center of said second portion, said first portion extending outwardly from each of said first lateral side and said second lateral side such that said first portion defines a pair of wings of said menstrual pad.

3. The assembly according to claim 2, wherein said absorbent member has a first end, a second end and an outer surface extending between said first end and said second end, said outer surface having a lower side, an upper side, a first sidelong side and a second sidelong side, said lower side being bonded to said top surface of said second portion, said absorbent member being elongated between said first end and said second end.

4. The assembly according to claim 3, wherein said absorbent member is oriented to extend along an axis extending between said front side and said back side of said perimeter edge of said second portion, said absorbent member being centrally positioned between said first lateral side and said second lateral side of said perimeter edge of said second portion, said second end of said absorbent member being positioned adjacent to said back side of said perimeter edge of said second portion, each of said first end and said second end of said absorbent member being rounded.

5. The assembly according to claim 3, wherein said ridge has a primary end, a secondary end and a lowermost surface extending between said primary end and said secondary end, said lowermost surface being bonded to said top surface of said second portion, each of said primary end and said secondary end abutting a respective one of said first sidelong side and said second sidelong side of said outer surface of said absorbent member at a point located adjacent to said second end of said absorbent member.

6. The assembly according to claim 5, wherein said ridge follows curvature of each of said first lateral side and said second lateral side of said perimeter edge of said second portion, said ridge forming a loop being positioned adjacent to said front side of said perimeter edge of said second portion.

7. A menstrual pad assembly for inhibiting leakage when a user is lying down, said assembly comprising:
a menstrual pad having a first portion being perpendicularly oriented with a second portion such that said menstrual pad forms a plus shape, said second portion having a top surface and a perimeter edge, said perimeter edge having a first lateral side, a second lateral side, a front side and a back side, said second portion being elongated between said front side and said back side, each of said first lateral side and said second lateral side curving inwardly toward a center of said second portion, said first portion extending outwardly from each of said first lateral side and said second lateral side such that said first portion defines a pair of wings of said menstrual pad;
an absorbent member extending upwardly from said second portion of said menstrual pad, said absorbent member being oriented to extend along a longitudinal axis of said second portion wherein said absorbent member is configured to extend between a user’s genitalia and the user’s buttocks when said menstrual pad is worn, said absorbent member being comprised of a fluid absorbent material wherein said absorbent member is configured to absorb bodily fluids thereby inhibiting the bodily fluids from escaping said menstrual pad, said absorbent member having a first end, a second end and an outer surface extending between said first end and said second end, said outer surface having a lower side, an upper side, a first sidelong side and a second sidelong side, said lower side being bonded to said top surface of said second portion, said absorbent member being elongated between said first end and said second end, said absorbent member being oriented to extend along an axis extending between said front side and said back side of said perimeter edge of said second portion, said absorbent member being centrally positioned between said first lateral side and said second lateral side of said perimeter edge of said second portion, said second end of said absorbent member being positioned adjacent to said back side of said perimeter edge of said second portion, each of said first end and said second end of said absorbent member being rounded; and
a ridge extending upwardly from said second portion of said menstrual pad, said ridge forming a closed loop wherein said ridge is configured to surround the user’s genitalia when said menstrual pad is worn, said ridge being comprised of a fluid absorbent material wherein said ridge is configured to inhibit bodily fluids from escaping said second portion thereby inhibiting the bodily fluids from staining clothing worn by the user, said ridge having a primary end, a secondary end and a lowermost surface extending between said primary end and said secondary end, said lowermost surface being bonded to said top surface of said second portion, each of said primary end and said secondary end abutting a respective one of said first sidelong side and said second sidelong side of said outer surface of said absorbent member at a point located adjacent to said second end of said absorbent member, said ridge following curvature of each of said first lateral side and said second lateral side of said perimeter edge of said second portion, said ridge forming a loop being positioned adjacent to said front side of said perimeter edge of said second portion.
